(19) United States
(12) Patent Application Publication
(10) Pub. No.: US 2021/0115398 A1
(43) Pub. Date: Apr. 22, 2021

(54) CORNEAL TISSUE CULTURE SYSTEM AND CORNEAL TISSUE CULTURE METHOD THEREOF

(71) Applicant: Chang Gung Memorial Hospital, Linkou, Taoyuan City (TW)

(72) Inventors: Hung-Chi CHEN, Taoyuan City (TW); Chao-Min CHENG, Hsinchu (TW); Chieh-Cheng HUANG, Hsinchu (TW); Yi-Jen HSUEH, Taoyuan City (TW)

(21) Appl. No.: 16/913,247

(22) Filed: Jun. 26, 2020

(30) Foreign Application Priority Data
Oct. 21, 2019 (TW) 108137947

(51) Int. Cl. C12N 5/079; C12M 1/22; C12M 1/12

(52) U.S. Cl. CPC C12N 5/0621; C12M 23/10; C12N 2513/00; C12N 2500/30; C12M 23/06

(57) ABSTRACT

A corneal tissue culture system and a corneal tissue culture method for increasing corneal endothelial cell density, healing of damaged corneal endothelial cell, and stimulating corneal endothelial cell proliferation are disclosed. The corneal tissue culture system comprises a tissue culture medium and a tissue culture dish to reduce metabolic pressure and reduce corneal tissue edema.

### CROSS REFERENCE TO RELATED APPLICATION

This application claims the benefits of the Taiwan Patent Application Serial Number 108137947, filed on Oct. 21, 2019, the subject matter of which is incorporated herein by reference.

### BACKGROUND OF THE INVENTION

### 1. Field of the Invention

The present invention relates to a corneal tissue culture system and a corneal tissue culture method, particularly, to a corneal tissue culture system and a corneal tissue culture method for increasing corneal endothelial cell density, healing of damaged corneal endothelial cell and stimulating corneal endothelial cell proliferation.

### 2. Description of Related Art

The lesion of the corneal tissue can be completely cured if the lesion is treated promptly and accurately in the early stage. But if the damage of the corneal tissue is too serious or the lesion of the corneal tissue is not treated in the early stage, the corneal transplantation must be performed to control the diseases. However, the number of the donor corneal tissue is rather low due to religious or traditional ideas. Accordingly, the number of corneal tissue donation is much lower than the demand for corneal tissue transplantation.

Please refer to FIG. 1, the corneal tissue is a transparent film having 5 layers and located on the front wall of the eyeball. The corneal tissue includes an epithelium layer 41, a lamina elastic anterior 42, a stroma layer 43, a lamina elastic anterior 44, and an endothelium layer 45. The endothelial cells of the corneal tissue are connected tightly with waterproof barrier function to prevent the aqueous humor flows into the extracellular space. Also, the endothelial cells of the corneal tissue have the pumping function to actively pump the water from the corneal stroma into the anterior chamber and maintain the thickness and transparency of the corneal tissue. However, the dysfunctional endothelial cells of the corneal tissue may cause corneal edema and lesions.

The endothelium layer of the corneal tissue is mainly composed of a monolayer of hexagonal flat endothelial cells, wherein the corneal endothelial cells are evenly and tightly arranged. However, the cell number of the endothelium layer will decrease with age and is not renewable. The defected area of the endothelium layer can only be filled by the expansion and migration of the adjacent cells. In contrast to corneal epithelial cells, human corneal endothelial cells are generally considered not expandable in vivo and slowly expandable in vitro. The endothelial cell density diminishes gradually from birth until death. The epithelium layer of the corneal tissue can be differentiated from limbal epithelial stein cells (LESC). Clinically, only donor corneas with an endothelial cell density greater than 2100 cells/mm² are eligible because the endothelial cell density is a critical indicator for postoperative follow-up and assessments.

The endothelial cell density is a key factor for maintaining the physiological function of the corneal tissue; therefore, damage of the endothelium layer of the corneal tissue, low endothelial cell density, or endothelium layer dysfunction may cause the corneal edema and the donated corneal tissue difficult to manipulate during the surgery or even unusable. However, the conventional method to preserve the donor corneal tissue fails to focus on how to stimulate the proliferation of the corneal endothelial cells. For example, the conventional corneal preservation solution Optisol GS is disadvantageous of high cost, and the longer the storage time, the more cells will be lost.

### SUMMARY OF THE INVENTION

In order to solve the aforementioned problems of lacking the donor corneal tissue, a technique for increasing the corneal endothelial cell density of the donor corneal tissue and restoring the endothelial cells of the declining corneal tissue is required to increase the availability of the donor corneal tissue to optimize the quality of the donor corneal tissue.

An object of the present invention is to provide a corneal tissue culture system, which is able to increase corneal endothelial cell density, heal damage corneal endothelial cells, and stimulate corneal endothelial cell proliferation.

Another object of the present invention is to provide a method of culturing a corneal tissue, wherein the corneal tissue is cultured in a specialized tissue culture dish allowing one portion of the corneal epithelium layer contact with air for simulating the corneal tissue in the real environment. The method of the present invention is effective in reducing the corneal edema; therefore, the quality of the donor corneal tissue may be improved.

The corneal tissue culture system of the present invention comprises a tissue culture media and a tissue culture dish, wherein the tissue culture media includes a basal medium and a lysophosphatidic acid (LPA), and the tissue culture dish accommodates the tissue culture media.

In one embodiment of the present corneal tissue culture system, the concentration of the lysophosphatidic acid in the tissue culture media is 10 μmol/L to 25 μmol/L, wherein 20 μmol/L is preferable.

In one embodiment of the present corneal tissue culture system, the basal medium is Dulbecco's modified Eagle's medium (DMED).

In one embodiment of the present corneal tissue culture system, the tissue culture media further includes a fetal bovine serum (FBS), wherein the weight percentage of FBS in the tissue culture media is 5%-20%, wherein 10% is preferable.

In one embodiment of the present corneal tissue culture system, the tissue culture media further includes an antibiotic agent, the antibiotic agent can be selected from those antibiotic agents known in the art. For example, the antibiotic agent can be at least one selected from the group consisting of gentamicin, penicillin, streptomycin, and mixture thereof, wherein gentamicin is preferable.

In one embodiment of the present corneal tissue culture system, the concentration of the antibiotic agent is 20-30 μg/mL, wherein 25 μg/mL of gentamicin is preferable.

In the present corneal tissue culture system, LPA is purchased from Sigma-Aldrich; DMEM, FBS, and gentamicin are purchased from Invitrogen.

In one embodiment of the present corneal tissue culture system, the tissue culture dish is a petri dish, wherein the petri dish is selected from any types of petri dish known in the art without limitation as long as the corneal tissue is able to be immersed in the tissue culture media.

In one embodiment of the present corneal tissue culture system, the tissue culture dish includes: a dish body having a first accommodating space and a first opening; and a tube body disposed in the first accommodating space, and having a second accommodating space and a second opening, wherein the second opening and the first opening face toward a first direction; wherein the tissue culture media is filled in the first accommodating space and the second accommodating space. In one embodiment, the tube body may be obtained by cutting down a portion of the 1000 μL micropipette. For example, 10 mm of the tube body may be cut from the 1000 μL micropipette and fixed at the center of the petri dish.

In one embodiment of the present corneal tissue culture system, a height of the tube body is smaller than a height of the dish body.

The method of culturing corneal tissue includes the steps of: (1) providing a tissue culture dish having a dish body and a tube body, wherein the dish body has a first accommodating space and a first opening, the tube body is disposed in the first accommodating space and has a second accommodating space and a second opening, the first opening and the second opening face toward a first direction; (2) filling a tissue culture media in the first accommodating space and the second accommodating space; and (3) disposing a corneal tissue on the second opening, wherein the corneal tissue has an epithelial cell layer facing the first direction, wherein at least a portion of the corneal tissue contacts with the tissue culture media.

In detail, please refer to the cross-sectional view of the corneal tissue illustrated in FIG. 1, the corneal tissue includes an epithelium layer 41, a lamina elastic anterior 42, a stroma layer 43, a lamina elastic anterior 44, and an endothelium layer 45. In the present invention, the corneal tissue 400 is placed in the tissue culture media with at least one portion of the epithelium layer 41 is exposed in the air for simulating the corneal tissue in the real environment.

According to one embodiment of the present method of culturing corneal tissue, in step (3), the at least a portion of the corneal tissue is at least a portion of the epithelial cell layer.

According to one embodiment of the present method of culturing corneal tissue, in step (3), the at least a portion of the corneal tissue is the corneal tissue as a whole, and the corneal tissue is immersed in the tissue culture media without contacting the air.

According to one embodiment of the present method of culturing corneal tissue, in step (1), a height of the tube body is smaller than a height of the dish body.

According to one embodiment of the present method of culturing corneal tissue, in step (2), the tissue culture media includes a basal medium and a lysophosphatidic acid, wherein the concentration of the lysophosphatidic acid in the tissue culture media is 10 μmol/L-25 μmol/L, wherein 20 μmol/L is preferable.

According to one embodiment of the present method of culturing corneal tissue, in step (2), the basal medium is DMEM.

According to one embodiment of the present method of culturing corneal tissue, in step (2), the tissue culture media further includes a fetal bovine serum, wherein the weight percentage of FBS in the tissue culture media is 5%-20%, wherein 10% is preferable.

According to one embodiment of the present method of culturing corneal tissue, the tissue culture media further includes an antibiotic agent, which is at least one selected from the group consisting of gentamicin, penicillin, streptomycin, and mixture thereof, wherein gentamicin is preferable.

According to one embodiment of the present method of culturing corneal tissue, in step (2), the concentration of the antibiotic agent is 20-30 μg/mL, wherein 25 μg/mL is preferable.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a cross-sectional view of the corneal tissue;

FIG. 2 shows the corneal tissue culture system of the first embodiment of the present invention;

FIG. 3 shows the corneal tissue culture system of the second embodiment of the present invention;

FIG. 4 shows the outer appearance of the corneal tissue cultured in the corneal tissue culture system of the present invention;

FIG. 5 shows the morphology of the corneal endothelial cell of the corneal tissue cultured in the corneal tissue culture system on the present invention;

FIG. 6 shows the cell number of the dose-response test of one embodiment of the present invention;

FIG. 7 shows the morphology of the corneal endothelium layer of the example and the comparison example of the present invention;

FIG. 8 shows the analysis diagram of the cell density of the corneal endothelium layer of the example and the comparison example of the present invention;

FIG. 9 shows the ZO-1 immunostaining cell morphology of the example and the comparison example of the present invention;

FIG. 10 shows the analysis result of the cell proliferation test of the example and the comparison example of the present invention;

FIG. 11 shows the analysis result of the cell proliferation test of the example and the comparison example of the present invention;

FIG. 12 shows the staining morphology of the damaged corneal tissue of the example of the present invention;

FIG. 13 shows the morphology of the corneal endothelium layer of the example and the comparison example of the present invention;

FIG. 14 shows another analysis diagram of the cell density of the corneal endothelium layer of the example and the comparison example of the present invention;

FIG. 15 shows the outer appearance and the cell morphology of the transplant corneal tissue of the example and the comparison example of the present invention;

FIG. 16 shows another analysis diagram of the cell density of the corneal endothelium layer of the example and the comparison example of the present invention;

FIG. 17 shows another ZO-1 immunostaining cell morphology of the example and the comparison example of the present invention;

FIG. 18 shows another analysis diagram of the cell density of the corneal endothelium layer of the example and the comparison example of the present invention;

FIG. 19 shows Ki-67 immunostaining cell morphology of the example and the comparison example of the present invention; and

FIG. 20 shows the test results of the example and the comparison example of the present invention.

### DETAILED DESCRIPTION OF THE PREFERRED EMBODIMENT

[Statistical Analysis]

All numeric results are shown as the mean±standard deviation. The data were compared using one-way ANOVA with Microsoft Excel version 2016 (Microsoft, Redmont, Wash.). The data were analyzed using two-tailed p values, where P<0.05* and P<0.01** were considered to indicate significant differences.

[Corneal Tissue Culture System]

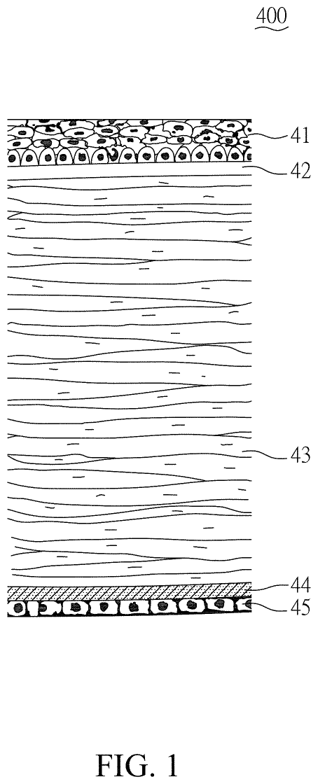

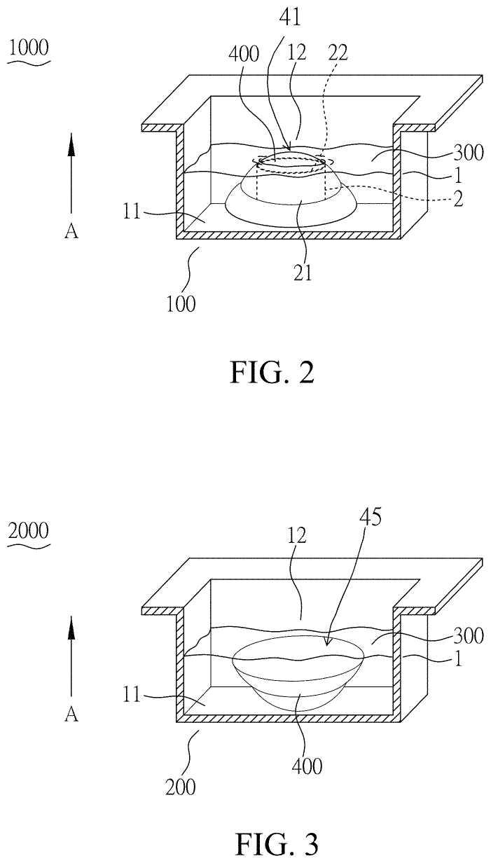

Please refer to the corneal tissue culture system 1000 of the first embodiment illustrated in FIG. 2, wherein the corneal tissue culture system 1000 comprises a tissue culture dish 100 and a tissue culture media 300. The tissue culture dish 100 includes a dish body 1 and a tube body 2, wherein the dish body 1 has a first accommodating space 11 and a first opening 12, the tube body 2 is disposed in the first accommodating space 11 and has a second accommodating space 21 and a second opening 22, wherein the first opening and the second opening face toward a first direction A. The tissue culture media 300 includes a Dulbecco's modified Eagle's medium (DMEM), a fetal bovine serum (FBS), gentamicin, and lysophosphatidic acid (LPA). In the present embodiment, the tissue culture media 300 is filled in the first accommodating space 11 and the second accommodating space 21. Please refer to FIG. 1, a corneal tissue 400 covers the second opening 22 with its epithelium layer facing the first direction A, wherein a portion of the corneal epithelium layer 41 contacts with the air and the corneal endothelium layer contact with the tissue culture media 300 for stimulating the real physiological environment of the anterior chamber.

Please refer to the corneal tissue culture system 2000 of the second embodiment of the illustrated in FIG. 3, wherein the corneal tissue culture system 2000 comprises tissue culture dish 200 and a tissue culture media 300. The tissue culture dish 200 includes a dish body 1, wherein the dish body 1 has a first accommodating space 11 and an opening 12 facing toward a first direction A. The tissue culture media 300 is filled in the first accommodating space 11. Please also refer to FIG. 1, the corneal tissue 400 is placed in the first accommodating space 11 and is immersed entirely in the tissue culture media 300 with the endothelium layer 45 facing the first direction A. The composition of the tissue culture media 300 is the same as the tissue culture media described in the first embodiment.

[The Observation of the Cultured Corneal Tissue]

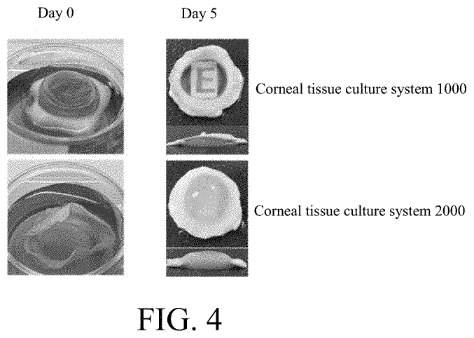

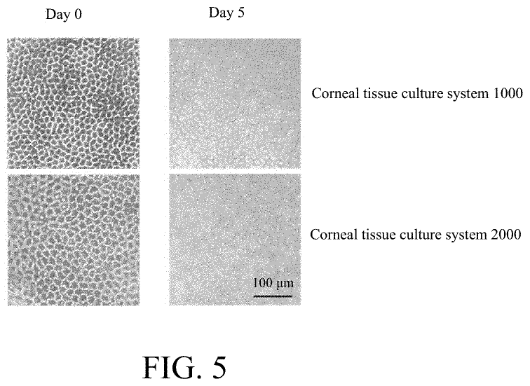

Firstly, a rabbit corneal tissue was placed in the tissue culture dish 100, cultivated with the tissue culture media 300 consisting of DMEM basal medium, 10% FBS, and 25 μg/mL gentamycin, and incubated at 37° C. with 5% CO₂, wherein a portion of the epithelium layer of the rabbit corneal tissue contacts with air (The tissue culture system 1000 as illustrated in FIG. 2). Another rabbit corneal tissue was placed in the tissue culture dish 200, immersed and cultivated with the same tissue culture media 300 (The tissue culture system 2000 as illustrated in FIG. 3). Gross appearances and transparency of the rabbit corneal tissue were observed after 5 days and were shown in FIG. 4. The morphology of the corneal endothelium layer was observed and shown in FIG. 5 (scale bar represent 100 μm). According to the results, the rabbit corneal tissue cultivated in the tissue culture dish 100 with a portion of the epithelium layer contacting the air was still had its transparency and the thickness thereof is 1281±110 μm. However, the rabbit corneal tissue immersed cultivated in the tissue culture dish 200 had lost its transparency; the thickness thereof was significantly increased from 379±9 μm to 3421±159 μm. Accordingly, the stromal edema of the rabbit corneal tissue cultivated in the tissue culture dish 100 with a portion of the epithelium layer contacting the air was significantly reduced comparing to that of the rabbit corneal tissue cultivated in tissue culture dish 200.

[Dose-Response Test]

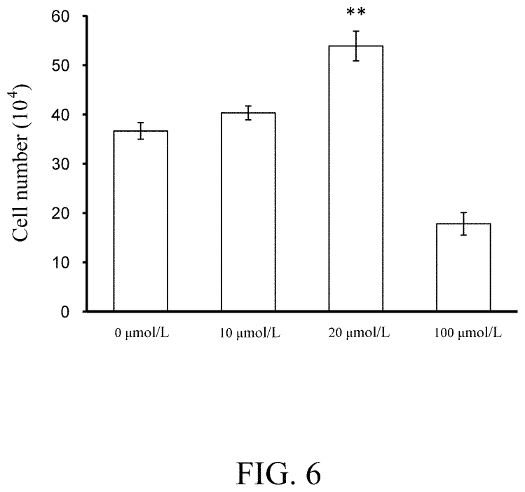

The tissue culture media (DMEM, 10% FBS, 25 μg/mL gentamycin) added with 0 μmol/L, 10 μmol/L, 20 μmol/L, and 100 μmol/L of LPA were used for co-cultivating the rabbit corneal endothelial cells and the rabbit corneal stroma cells at 37° C. with 5% CO₂. After 5 days of cultivation, the cell number of the rabbit corneal endothelial cells of each groups were calculated and shown in FIG. 6. The result showed that the tissue culture media added with 20 μmol/L of LPA significantly stimulated corneal endothelial cell proliferation and improved the corneal endothelial cell density. According to the result, the tissue culture media used for the following experiments was added with 20 μmol/L LPA.

[Evaluation of the Corneal Endothelial Cell Density of the Corneal Tissue]

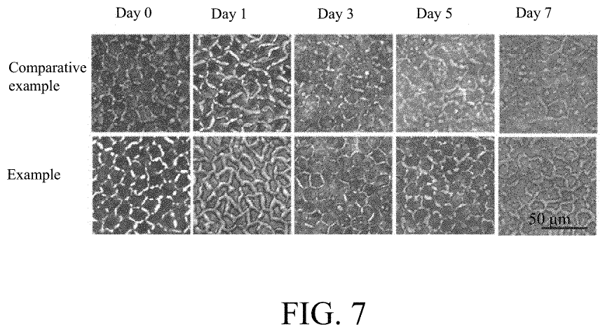

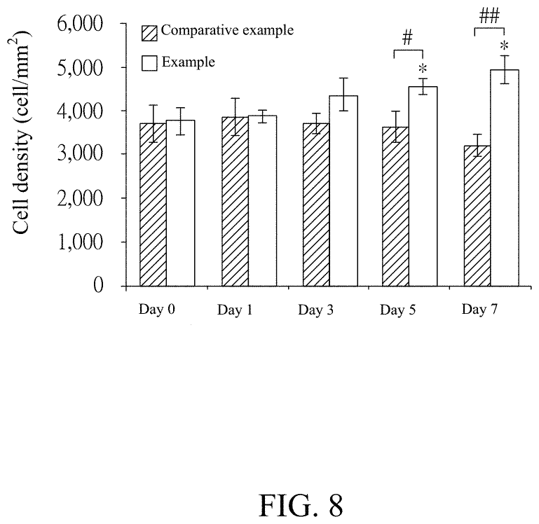

The rabbit corneal tissues were placed individually in the tissue culture dishes 100, cultivated with the tissue culture media (DMEM basal medium, 10% FBS, and 25 μg/mL gentamycin) added with 20 μmol/L LPA (Example) or without LPA (Comparative example), and incubated at 37° C. with 5% CO₂, wherein a portion of the corneal epithelium layer of the rabbit corneal tissues faced toward the first direction A and contacted with air and the corneal endothelium layer contacted with the tissue culture media. After 7 days of cultivation, the cell morphology and the analysis of cell density of the corneal endothelium layer were shown in FIG. 7 and FIG. 8. The density of the corneal endothelial cells of the example on day 0 was 3766.7±332.9 cells/mm², 4572.2±179.8 cells/mm² on day 5, and 4950.0±304.1 cells/mm² on day 7. However, the density of the corneal endothelial cells of the comparative example showed no significant changes on day 0 to day 7, wherein the density of the corneal endothelial cells of the comparative example on day 5 was 3616.7±354.7 cells/mm² and 3200.0±259.8 cells/mm² on day 7. According to the result, the corneal endothelial cell density may be increased in the LPA-treated example.

[Morphology of the Corneal Endothelial Cells of the Corneal Tissue]

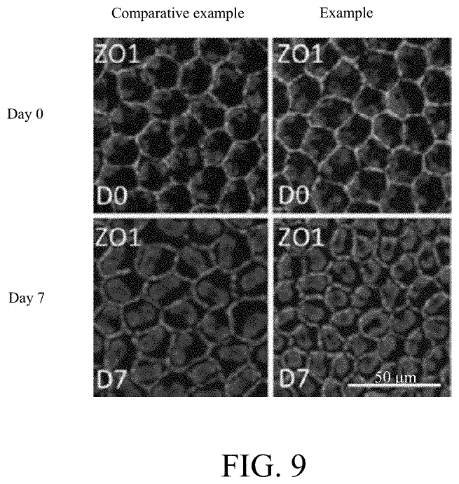

The rabbit corneal tissues were placed individually in the tissue culture dishes 100, cultivated with the tissue culture media (DMEM basal medium, 10% FBS, and 25 μg/mL gentamycin) added with 10 μmol/L LPA (Example) or without LPA (Comparative example), and incubated at 37° C. with 5% CO₂ for 7 days, wherein a portion of the corneal epithelium layer of the rabbit corneal tissues faced toward the first direction A and contacted with air and the corneal endothelium layer contacted with the tissue culture media. The corneal endothelium layer was immunostained for ZO-1 on day 0 and day 7 for observing the cell morphology of the corneal endothelial cells of the corneal tissue. The results shown in FIG. 9 showed a normal hexagonal phenotype (refer to the green area). The results revealed that the morphology of the corneal endothelial cells was not altered in tissue cultivation treated with LPA.

[Evaluation of the Cell Proliferation of the Corneal Tissue]

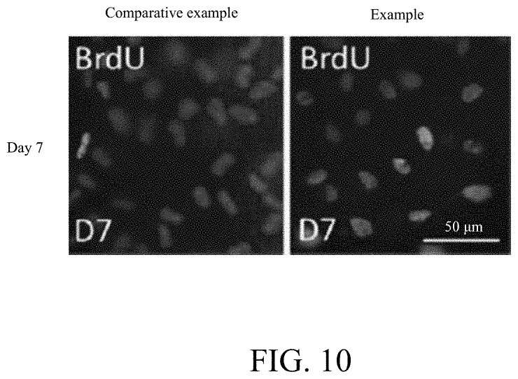

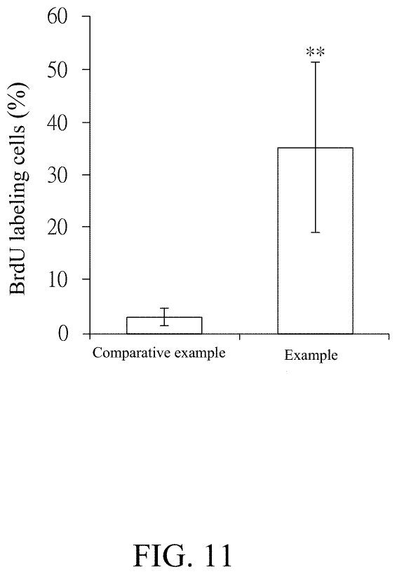

The rabbit corneal tissues were placed individually in the tissue culture dishes 100, cultivated with the tissue culture media (DMEM basal medium, 10% FBS, and 25 μg/mL gentamycin) added with 10 μmol/L LPA (Example) or without LPA (Comparative example), and incubated at 37° C. with 5% CO₂ for 7 days, wherein a portion of the corneal epithelium layer of the rabbit corneal tissues faced toward the first direction A and contacted with air and the corneal endothelium layer contacted with the tissue culture media. Bromodeoxyuridine (BrdU) was added to the culture media from day 5 to day 7 for observing the proliferation of the cells. The BrdU labeling cells (green), shown in FIG. 10, were significantly increased in the LPA-treated example comparing to that of the comparative example. The percentage of the proliferated cells were shown in FIG. 11, wherein the BrdU labeling cells was 35.2%±16.1% in the example and was 2.8%±1.6% in the comparative example. According to the results, the corneal endothelial cells were significantly increased in the LPA-treated example.

[Investigation of the Healing Effect of the Damaged Corneal Endothelium Layer of the Corneal Tissue]

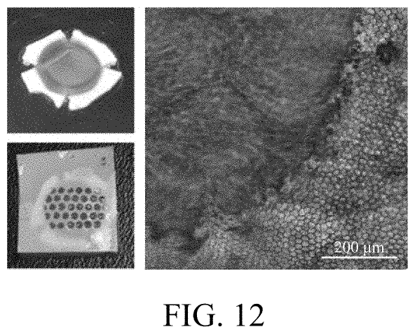

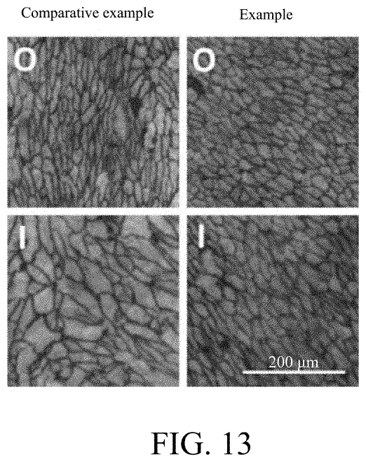

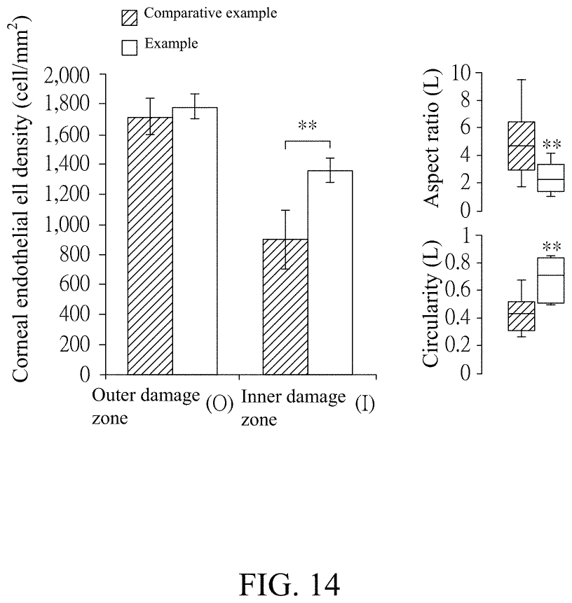

A polyvinylidene fluoride (PVDF) filter membrane with regularly punched-out holes was used to cover the rabbit corneal endothelium layer; exposing the inner damage zone (I) scraped with Cellulose Eye Spears. The rest area of the corneal endothelium layer was defined as the outer damage zone (O). The damaged corneal tissues were placed individually in the tissue culture dishes 100, cultivated with the tissue culture media (DMEM basal medium, 10% FBS, and 25 μg/mL gentamycin) added with 20 μmol/L LPA (Example) or without LPA (Comparative example), and incubated at 37° C. with 5% CO₂, wherein a portion of the corneal epithelium layer of the rabbit corneal tissues faced toward the first direction A and contacted with air and the corneal endothelium layer contacted with the tissue culture media. The inner damage zone (I) and the outer damage zone (O) of the corneal tissues on day 0 and day 2 were observed using Alizarin Red S staining. The morphology of the corneal tissue stained by Alizarin Red S was shown in FIG. 12, wherein the stained area (brown color) represented the corneal endothelial cells of the inner damage zone (I) and the unstained area represented the corneal endothelial cells of the outer zone (O). Please refer to FIG. 13, which showed that the inner damage zone (I) of the example was repopulated by neighbor corneal endothelial cells two days after tissue cultivation, but the cell density of the inner damage zone (I) of the comparative example was lower than that of the example. Refer to FIG. 14, the corneal endothelial cell density of the inner damage zone of the LPA-treated example was 1358±8 0.4 cells/mm², and the circularity and aspect ratio of the corneal endothelial cells were 0.68±0.15 and 2.39±1.14. However the corneal endothelial cell density of the inner damage zone of the comparative example was 900.0±198.4 cells/mm², and the circularity and aspect ratio of the corneal endothelial cells were 0.43±0.14 and 4.91±2.43. According to the results, the addition of LPA can stimulate cell proliferation of the damaged corneal tissue. Also, LPA enhanced the repopulation of the neighbor corneal endothelial cells to the damage zones.

[Evaluation of Corneal Tissue Transplantation]

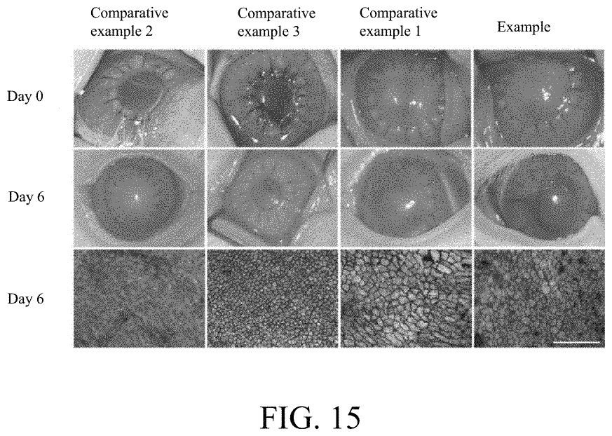

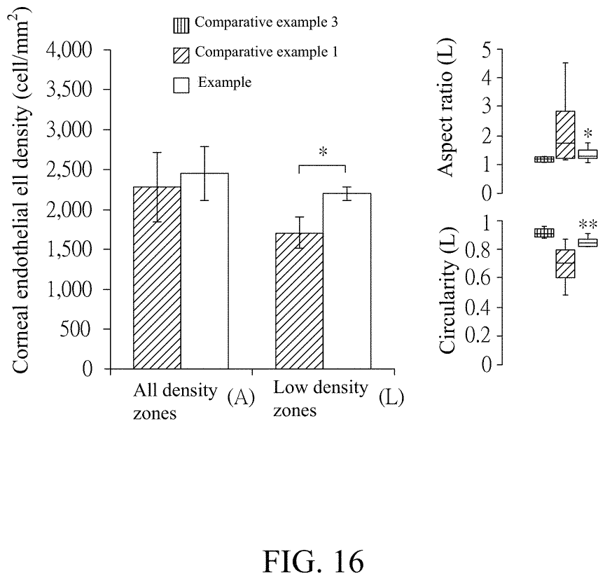

The damaged corneal tissues were prepared using the PVDF filter membrane described above (please refer to the above paragraph). Then, those damaged corneal tissues were placed individually in the tissue culture dishes 100, cultivated with the tissue culture media (DMEM basal medium, 10% FBS, and 25 μg/mL gentamycin) added with 20 μmol/L LPA (Example) or without LPA (Comparative example 1), and incubated at 37° C. with 5% CO₂, wherein a portion of the corneal epithelium layer of the rabbit corneal tissues faced toward the first direction A and contacted with air and the corneal endothelium layer contacted with the tissue culture media. After 2 days of cultivation, the damaged corneal tissues were transplanted into rabbit eyes. In addition, total corneal endothelium scrapping and undamaged mock donor corneas were also prepared to serve as comparative examples 2 and 3. External eye photography was taken in the rabbits after transplantation on day 0 and day 6, respectively. Following sacrifice on day 6, the corneal endothelial cells were stained with Alizarin Red S for observations of the cell morphology of the corneal endothelial cells in all density zones (A) (average of high-density and low-density zones) and cell shape in low-density zone (L) were examined. The results were shown in FIG. 15, wherein the corneal tissues of the example and the comparative example 3 remained transparent, but the corneal tissue of the comparative example 1 became turbid. Please refer to FIG. 16 showing the analysis of the corneal endothelial cell density, circularity, and aspect ratio. The corneal endothelial cell density of the example was 2200.0±90.1 cells/mm², and the circularity and the aspect ratio of the corneal endothelial cells of example 1 were 0.84±0.05 and 1.35±0.02. However, in comparative example 1, the cell density was 1700.0±288.3 cells/mm², the circularity was 0.69±0.12, and the aspect ratio was 2.12±1.13. According to the results, the corneal endothelial cell density in the LPA-treated example was significantly increased and the transparency of the corneal tissue had remained, that is, the wound healing effect was achieved.

[Gene Expression Analysis Using DNA Microarray]

The gene expression profiling and the cytokine of the human corneal stromal cells were analyzed using cDNA microarray chip and Cytokine Array G5 chip. The human corneal stromal cells were cultured with culture media (DMEM basal medium, 10% FBS, and 25 μg/mL gentamycin) added with 20 μmol/L LPA (served as an Example) or without adding LPA (Comparative example) and incubated at 37° C. with 5% CO₂. After 2 days of cultivation, the total RNA from the human corneal stromal cells in example and comparative example were isolated with the TRIzol reagent and purified using RNeasy column. After amplification and labeling the total RNA samples, GeneChip hybridization was performed and a GeneArray G7 scanner was used to scan the results. The culture media from the example and the comparative example were collected and the cytokines were analyzed using a human cytokine antibody array. The results were shown in Table 1, wherein CCL20 and IL-1β (IL1B) gene expressions were significantly up-regulated out of 16 genes with high expressions in the LPA-treated example. Refer to Table 2., CCL20 and IL-1β (IL1B) cytokines also showed higher secretion in the LPA-treated example. Accordingly, the secretion of CCL20 and IL-1β were significantly increased when treated with LPA. It was confirmed that LPA stimulated the proliferation of the endothelial cells of the corneal tissue via two pathways including directly affecting the endothelial cells and stimulating the secretion of CCL20 and IL-1β from the endothelial cells.

TABLE 1
Overexpressed secreted genes with fold change >10

| Gene | | Gene expression in |
|---|---|---|
| symbol | Description | stromal cells |
| CCL20 | Chemokine (C-C motif) ligand 20 | 625.99 |
| IL1B | Interleukin 1 beta | 572.05 |
| CSF3 | Colony stimulating factor 3 | 85.04 |
| 1L1A | Interleukin 1 alpha | 58.49 |
| OLR1 | Oxidized low density lipoprotein | 24.25 |
| | receptor 1 | |
| CXCL5 | Chemokine (C-X-C motif) ligand 5 | 22.32 |
| CXCL10 | Chemokine (C-X-C motif) ligand 10 | 20.97 |
| INHBA | Inhibin beta A | 20.97 |
| ESM1 | Endothelial cell-specific molecule 1 | 20.11 |
| CCL5 | Chemokine (C-C motif) ligand 5 | 17.63 |
| 1L1RN | Interleukin 1 receptor antagonist | 17.39 |
| CCL8 | Chemokine (C-C motif) ligand 8 | 15.89 |
| 1L7R | Interleukin 7 receptor | 15.03 |
| CCL7 | Chemokine (C-C motif) ligand 7 | 11.39 |
| CXCL3 | Chemokine (C-X-C motif) ligand 3 | 11.31 |
| EPGN | Epithelial mitogen | 10.85 |

TABLE 2
LPA mediates corneal stromal cell-derived expression
and secretion of cytokines (fold change)

| Gene | | Gene expression in | Cytokine content |
|---|---|---|---|
| symbol | Description | stromal cells | in media |
| CCL20 | Chemokine (C-C | 625.99 | 3.26 |
| | motif) ligand 20 | | |
| IL1B | Interleukin 1 beta | 572.05 | 1.73 |
| CSF3 | Colony stimulating | 85.04 | 0.85 |
| | factor 3 | | |
| IL1A | Interleukin 1 alpha | 58.49 | 0.80 |

[Evaluation of the Proliferation and Morphology of the Corneal Endothelial Cells and of the Corneal Tissue]

The cell morphology, cell density, and cell proliferation of the present evaluation were performed through immunostaining.

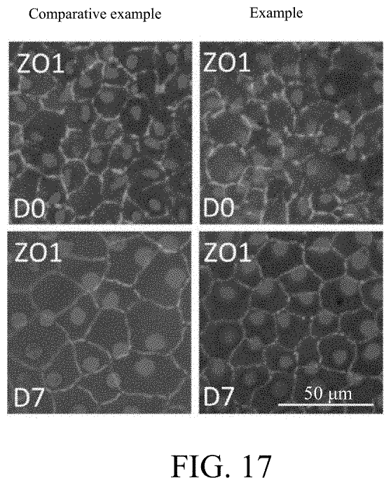

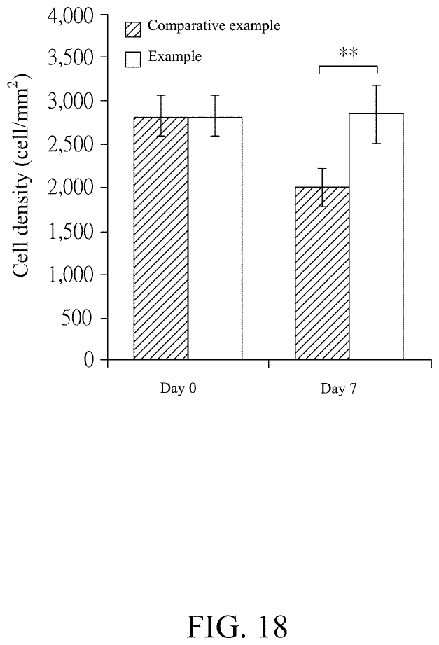

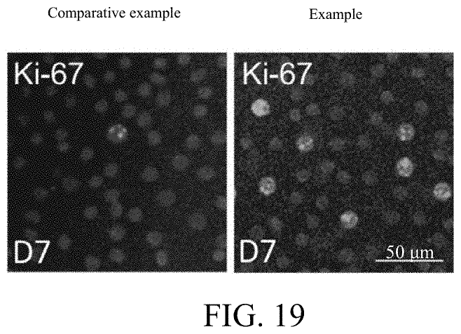

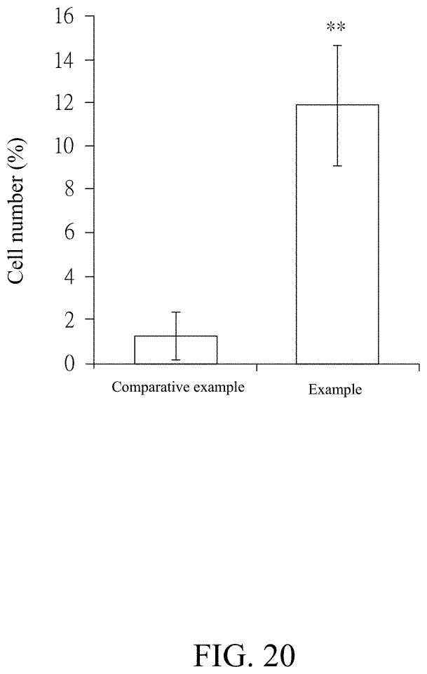

One-third of the human corneal residual tissue samples were used for immunostaining for ZO-1 directly (Day 0), while the remaining two-thirds were immersed and placed endothelial side up in the tissue culture media (DMEM basal medium, 10% FBS, and 25 μg/mL gentamycin) added with 20 μmol/L LPA (served as an Example) or without adding LPA (Comparative example) and incubated at 37° C. with 5% CO₂. 7 days later, the tissues were cut into halves and subjected to immunostaining for ZO-1 and Ki-67 to evaluate the effects of LPA treatment on corneal endothelial cell density and cell proliferation. As shown in FIG. 17 and FIG. 18, the corneal endothelial cell density was significantly lower in the comparative example than in the LPA-treated example on Day 7 Immunostaining for ZO-1 revealed that the morphology of corneal endothelial cells was not altered in the example or the comparative example and remained the normal hexagonal phenotype. The corneal endothelial cell density of the LPA-treated example and the comparative example were 2848.8 cells/mm²±331.7 cells/mm² and 2004.7 cells/mm²±220.2 cells/mm². Additionally, please refer to FIG. 19 showing the Ki-67 immunostaining cell morphology, more Ki-67-positive cells (light blue) were observed in the LPA-treated example than in the comparative example. Please also refer to the cell number analysis shown in FIG. 20, wherein Ki-67-positive cells of the LPA-treated example was 11.86%±2.80%, while the Ki-67-positive cells of the comparative example was 1.25%±1.09%. The results indicated that corneal endothelial cell density and cell proliferation were enhanced after LPA treatment without affecting the cell morphology.

Base on the aforementioned experimental results, the corneal tissue culture system of the present invention is advantageous for increasing corneal endothelial cell density, healing of damaged corneal endothelial cell, and stimulating corneal endothelial cell proliferation by adding LPA.

What is claimed is:

1. A corneal tissue culture system, comprising:
a tissue culture media including a basal medium and a lysophosphatidic acid; and
a tissue culture dish accommodating the tissue culture media.

2. The corneal tissue culture system as claimed in claim 1, wherein the concentration of the lysophosphatidic acid in the tissue culture media is 10 μmol/L to 25 μmol/L.

3. The corneal tissue culture system as claimed in claim 2, wherein the concentration of the lysophosphatidic acid in the tissue culture media is 20 μmol/L.

4. The corneal tissue culture system as claimed in claim 1, wherein the basal medium is Dulbecco's modified Eagle's medium (DMED).

5. The corneal tissue culture system as claimed in claim 1, wherein the tissue culture media further includes a fetal bovine serum (FBS), wherein the weight percentage of FBS in the tissue culture media is 5%-20%.

6. The corneal tissue culture system as claimed in claim 1, wherein the tissue culture media further includes an antibiotic agent, the antibiotic agent is at least one selected from the group consisting of gentamicin, penicillin, streptomycin, and mixture thereof.

7. The corneal tissue culture system as claimed in claim 6, wherein the concentration of the antibiotic agent is 20-30 μg/mL.

8. The corneal tissue culture system as claimed in claim 1, wherein the tissue culture dish is a petri dish.

9. The corneal tissue culture system as claimed in claim 1, wherein the tissue culture dish includes:
a dish body having a first accommodating space and a first opening; and
a tube body disposing in the first accommodating space and having a second accommodating space and a second opening, wherein the second opening and the first opening face toward a first direction;
wherein the tissue culture media is filled in the first accommodating space and the second accommodating space.

10. The corneal tissue culture system as claimed in claim 9, wherein a height of the tube body is smaller than a height of the dish body.

11. A method of culturing corneal tissue, including the steps of:
(1) providing a tissue culture dish having a dish body and a tube body, wherein the dish body has a first accommodating space and a first opening, the tube body is disposed in the first accommodating space and has a second accommodating space and a second opening, the first opening and the second opening face toward a first direction;
(2) filling a tissue culture media in the first accommodating space and the second accommodating space; and
(3) disposing a corneal tissue on the second opening, wherein the corneal tissue has an epithelial cell layer facing the first direction, wherein at least a portion of the corneal tissue contacts with the tissue culture media.

12. The method as claimed in claim 11, wherein step (3), the at least a portion of the corneal tissue is at least a portion of the epithelial cell layer.

13. The method as claimed in claim 11, wherein step (3), the at least a portion of the corneal tissue is the corneal tissue as a whole.

14. The method as claimed in claim 11, wherein step (1), a height of the tube body is smaller than a height of the dish body.

15. The method as claimed in claim 11, wherein step (2), the tissue culture media includes a basal medium and a lysophosphatidic acid.

16. The method as claimed in claim 15, wherein the concentration of the lysophosphatidic acid in the tissue culture media is 10 μmol/L-25 μmol/L.

17. The method as claimed in claim 16, wherein the concentration of the lysophosphatidic acid in the tissue culture media is 20 μmol/L.

18. The method as claimed in claim 15, wherein step (2), the basal medium is DMEM.

19. The method as claimed in claim 15, wherein step (2), the tissue culture media further includes a fetal bovine serum, wherein the weight percentage of FBS in the tissue culture media is 5%-20%.

20. The method as claimed in claim 15, wherein step (2), the tissue culture media further includes an antibiotic agent, which is at least one selected from the group consisting of gentamicin, penicillin, streptomycin, and mixture thereof.

21. The method as claimed in claim 20, wherein step (2), the concentration of the antibiotic agent is 20-30 μg/mL.
